(19) United States
(12) Patent Application Publication
(10) Pub. No.: US 2002/0150618 A1
(43) Pub. Date: Oct. 17, 2002

(54) Process for preparing solid dosage forms of very low-dose drugs

(75) Inventors: James Albert Napper, Pound Hill (GB); Karen Triona O'Brien, Bishops Stortford (GB); Sultan James Manek, Epsom (GB); Rajinder Kumar, Cambridge (GB); Julia Mary Loudon, Buntingford (GB); Michael Sydney George Clark, Much Hadham (GB); Neil Mortimer, Woking (GB)

Correspondence Address: GLAXOSMITHKLINE, Corporate Intellectual Property - UW2220, P.O. Box 1539, King of Prussia, PA, 19406-0939, US

(73) Assignee: SmithKline Beecham p.l.c.

(21) Appl. No.: 10/167,319

(22) Filed: Jun. 10, 2002

Related U.S. Application Data
(63) Continuation of application No. 09/759,575, filed on Jan. 12, 2001, now abandoned.
Application No. 09/759,575 is a continuation of application No. 09/000,252, filed on Jul. 8, 1998, now abandoned.
Application No. 09/000,252 is a 371 of international application No. PCT/EP96/03326, filed on Jul. 29, 1996.

(30) Foreign Application Priority Data
Jul. 29, 1995 (GB) 9515624.6
Mar. 29, 1996 (GB) 9606684.0

(51) Int. Cl. A61K 9/20; A61K 31/407

(52) U.S. Cl. 424/465; 514/412

(57) ABSTRACT

A drug formulation process which comprises admixing carrier particles with a solution of drug in water in a quantity of 1-3% by weight of solution to total mix and a method of treatment and/or prophylaxis of dementia, which method comprises administering to the patient [R-(Z)]-α-(methoxyimino)-α-(1-azabicyclo [2.2.2]oct-3-yl)acetonitrile monohydrochloride at a daily dose below 0.01 mg/kg and pharmaceutical compositions used therein.

This invention relates to a method of formulating solid dosage forms of drugs and to solid dosage forms produced thereby, in particular solid dosage forms containing low dose of drug. The invention also relates to a method of treatment and/or prophylaxis of dementia and unit dosage forms useful therein.

The usual challenge in manufacturing solid dosage forms of very low-dose drugs, for example with active doses around 5-125 microgramme (μg) (for example 0.004-0.1% by weight of drug to total solid), is to ensure homogeneity. The technical problem is how to distribute the drug substance evenly among the large amount of excipient particles.

The simplest way of manufacturing tablets is simply to blend all the ingredients as dry powders and tablet them (“direct compression”). This is rarely successful for low-dose drugs; a common problem being segregation of the powder blend during tabletting. A variation of this method which has been successful for low-dose drugs is known as “trituration”, and is sometimes referred to as “ordered mixing” or “interactive mixing”. Very fine particles of the drug are first mixed with a small portion of excipient; the product then mixed with a slightly larger portion of excipient and so on until the desired mix is obtained. This method relies on the fine drug particles adhering electrostatically to the larger excipient ones, thus preventing segregation. The method works with some drugs, but success depends on the surface properties of both drug and excipient, and the method is very laborious. EP0503521 describes the application of this method to steroidal drugs with high binding affinity and low demixing potential for certain excipients.

A preferred alternative method for formulating low dose drugs is known as “wet granulation”. The drug is dissolved in water or another solvent, and blended with excipients including a binder, for example povidone, to form a wet mass containing 5-20% by weight of solution to total weight of granulation mix, which is then dried off in a separate step. The binder causes particles of excipient to clump together, and as the mass dries these clumps (“granules”) either contain or are coated with the drug. This is effective but cumbersome since the drying step requires special equipment, and generally involves high temperatures which may degrade labile drugs. Also, the use of the binder requires the further inclusion of a disintegrant such as sodium starch glycolate or starch to help the tablet, which is cohesive, to disperse in the stomach.

Fluid bed granulation has been used to achieve content uniformity of low dose (1 μg-10 mg) tablets (Thiel et al., J. Pharm. Pharmacol. 1986, 38, 335-343). In this process, the micronised drug is blended as a powder with other excipients, then loaded into a fluid bed granulator, and the powders are agglomerated by spraying on a solution of a binder, drying takes place concomitantly. The process does not require a separate drying step, but it does require the use of micronised drug, and also incorporates a separate blending step prior to granulation. It also requires specialised equipment and precise optimisation of the process parameters.

Another process for formulating low dose drugs is known as carrier granulation (Michoel et al., Pharmaceutical Technology June 1988, 66-84). This functions by spraying a solution of binder such as povidone in water onto relatively large excipient particles such as hydrous lactose and then spraying small dry drug substance particles onto that, thus coating the excipient with drug particles which are stuck on by the binder. The quantity of solution used was 3.3-3.5% by weight of solution to total granulation mix. The method was applied to a formulation containing 4-5% drug by weight. This method also requires drying; the drug particle size needs to be very small, which often requires an extra milling step and the very fine drug powder may not flow at all well; and the formula still requires a disintegrate.

Dahl et al. Drug Development and Industrial Pharmacy 1990, 16 (12), 1881-1891, describes the preparation of solid capsule formulations using a spray-on liquid drug carrier. The model drug is dissolved in a non-volatile solvent, propylene carbonate, and sprayed onto a compressible sugar at a loading of around 0.01% by weight of drug to total solid, to give a final unit dose of 35 μg. The solvent, being non-volatile, remains in the blend. It is added at around 5% by weight of the total formulation; lower ratios of solvent to solid resulted in decreased ability to disintegrate and dissolve. The resulting, somewhat sticky, powder showed some difficulties in automated encapsulation machines, and would be likely to give sight problems in tabletting.

Yalkowski (U.S. Pat. No. 4,489,026) describes a process which involves very slowly spraying a dilute solution of drug in a volatile inert solvent, preferably an organic solvent having a boiling point lower than 80° C., onto excipient powder in an open coating pan; a continuous flow of air dries the product during the spraying process. This process was applied to drugs with a unit dose of 10 μg or less. The spray rate is limited to 1-10 ml/min, making the process suitable only for very small batch-sizes (the example quoted prepared 1000 tablets). The weight ratio of solution to carrier used was 15%; also, the use of volatile organic liquids is now regarded as a significant hazard, requiring solvent-recovery processes and explosion-proof equipment.

Katdare (U.S. Pat. No. 4,898,736) describes a simplified version of this process, suitable for unit doses of 50-1000 μg; the drug, dissolved in an easily evaporated solvent such as ethanol, methanol, acetone or tetrahydrofuran, is simply blended with excipients in a ratio of 2.26% or 6.75% and then dried, followed by lubrication and tabletting. This process is in principle suitable for commercial scale manufacture, but does still have the problems associated with the use of volatile organic solvents.

According to the present invention there is provided a drug formulation process which comprises admixing carrier particles with a solution of drug in water in a quantity of 1-3% by weight of solution to total mix.

The resulting mixture may be formulated into suitable unit dose presentation, for example by tabletting and optionally film coating the tablets or by encapsulation.

It may be convenient to make the initial drug/carrier mix with a higher drug concentration, and then in a separate step blending that with further carrier, and this may be particularly useful where a range of tablet strengths is required. The separate blending step aids drying by the dilution effect, that is, the residual water is distributed through a greater quantity of carrier powder, and by the longer mixing time. The dilution is conveniently in the range 4/1 to 40/1 carrier/concentrate by weight, depending upon the processing characteristics of the carrier. A convenient dilution ratio for lactose monohydrate is 10/1.

Where the dilution step is not employed, the solution/mix weight ratio is more preferably up to 2% by weight

The optimum quantity of solution will depend upon the absorbent qualities of the carrier particles, the solubility of the drug and the characteristics of the mixing device, the quantity of solution being chosen so as to allow even distribution of drug while avoiding the need for a heated drying step.

The mixing step is preferably carried out in a high shear mixer.

The carrier may be any suitable soluble, directly compressible pharmaceutically acceptable excipient such as anhydrous lactose, lactose monohydrate, mannitol, or an insoluble, directly compressible pharmaceutically acceptable excipient such as microcrystalline cellulose or dicalcium phosphate, preferably a soluble excipient.

Any drug having a sufficient degree of solubility in water may be formulated by the process of the invention. The concentration of drug in the solution is dependent on the unit dose of drug required, the upper limit being dependent on the solubility of the drug.

During mixing, the carrier particles are evenly coated with a very thin film of drug solution. Some of the water naturally dries off during the mixing since there is normally a small airflow through the mixer; the remaining amount is so low that drying is not specifically required. If the tablets are film-coated, a degree of further drying may be obtained during the coating process.

The process of the invention has a number of advantages:

it does not require any milling of the drug substance

there is no need for a drying step. This simplifies processing and reduces production costs; heat labile drugs do not suffer at the temperature required for drying; for drugs which are highly potent, the omission of the drying step makes it easier to contain dust, improving safety for the factory worker

the use of volatile organic solvents is avoided

there is no need to add a binder

there is no need to add a disintegrant when the excipient is a highly soluble one.

Optional additives in the final mix include a disintegrant; a range of acidic or alkaline excipients to improve chemical stability of the drug in the formulation such as sodium dihydrogen citrate, preferably included in the initial mix; a lubricant such as magnesium stearate; and a glidant such as colloidal silica.

The present invention further provides a pharmaceutical composition comprising a drug formulated in accordance with the process of the invention and the use of said composition as an active therapeutic substance.

The invention additionally provides a pharmaceutical composition comprising a drug, obtainable by the process of the invention and the use of said composition as an active therapeutic substance.

The process of the invention is particularly useful for formulating [R-(Z)]-α-(methoxyimino)-α-(1-azabicyclo [2.2.2]oct-3-yl)acetonitrile monohydrochloride (compound X) with active doses around 5-125 microgramme (μg). Compound X and methods for its preparation are disclosed in EP-A-0392803, WO95/31456 and WO93117018. The compound enhances acetylcholine function via an action at muscarinic receptors within the central nervous system, and is therefore of potential use in the treatment and/or prophylaxis of dementia in mammals.

In particular the invention provides a pharmaceutical composition comprising a pharmaceutically acceptable carrier and compound X at a level of up to 0.1% by weight of drug to carrier, 0% volatile organic solvent and 0% binder.

EP-A-0392803 suggests the suitable daily dose for compound X and other compounds disclosed therein as 0.01-5 mg/kg. It has been surprisingly found through administration to human patients that efficacy as a cognition enhancer may be obtained at daily doses below 0.01 mg/kg more particularly 0.003 mg/kg and below, for example 0.0001-0.003 mg/kg, such as 0.00035-0.003 mg/kg, 0.0007-0.003 mg/kg, 0.0001-0.0007 mg/kg or 0.00035-0.002 mg/kg.

Accordingly the present invention provides a method of treatment and/or prophylaxis of dementia, and more particularly a method of enhancing cognition in a patient, which method comprises administering to the patient compound X at a daily dose below 0.01 mg/kg, more preferably 0.003 mg/kg or less. The invention also relates to the use of compound X in the manufacture of a medicament for the treatment and/or prophylaxis of dementia at a daily dose below 0.01 mg/kg. more preferably of 0.003 mg/kg or less. The invention further relates to a pharmaceutical composition for the treatment and/or prophylaxis of dementia which comprises compound X at a unit dose suitable for administration at a daily dose below 0.01 mg/kg, more preferably of 0.003 mg/kg or less, and a pharmaceutically acceptable carrier.

Suitable unit doses to achieve such daily doses are 5, 12.5, 25, 50 or 75 μg, administered twice daily and, in the case of 50 μg, once daily. Such unit doses are calculated on the basis of 50-70 kg individuals. The invention extends to the method, use or composition defined above wherein compound X is provided in such unit doses

The invention further provides a pharmaceutical composition comprising compound X of the invention and/or formulated in accordance with the process of the invention, in unit dose form selected from the range 5-125 μg per unit dose, such as 5, 12.5, 25, 50 or 75 μg per unit dose and the use of said composition as an active therapeutic substance, in particular in the treatment and/or prophylaxis of dementia.

### EXAMPLE

### Formulation of Compound X

To make 100 kg of blend for tabletting or encapsulation, at 100 ug drug per 150 mg excipient:

dissolve 67 g drug in 1 liter of water (i.e. 1% of water (1.067% of solution) on a weight basis)

slowly add this to 100 kg of a “direct compression” grade of lactose monohydrate in a high-shear mixer-granulator

blend with 0.25 kg lubricant (Magnesium Stearate) and 0.25 kg Glidant (Colloidal Silica)

tablet

filmcoat (optional)

To make 100 kg of blend for tabletting or encapsulation, at 100 ug drug per 150 mg excipient, by way of a concentrate:

dissolve 67 g drug in 0.1 liter of water

slowly add this to 9.8 kg, of a “direct compression” grade of lactose monohydrate and 0.2 kg of acidifying agent (Sodium Dihydrogen Citrate) in a high-shear mixer-granulator, followed by a further 0.1 liter of water to rinse the containers used, while mixing vigorously with a chopper speed of around 1500 rpm and then raising the speed to around 3000 rpm, for 10-20 minutes total mixing time (i.e. a total of 2% of water (2.67% of solution) is added on a weight basis relative to the amount of concentrate)

sieve the resulting concentrate into a tumble-blender

blend with 88 kg father lactose and 1.8 kg Sodium Dihydrogen Citrate, then blend in 0.15 kg Glidant (Colloidal Silica) and 0.25 kg lubricant (Magnesium Stearate)

tablet

filmcoat (optional)

To make unit doses of 75, 50, 25, 12.5 and 5 μg in 150 mg excipient, the amount of drug used in the above methods is 50, 33.6, 16.8. 8.4 and 3.3 g respectively.

1. A drug formulation process which comprises admixing carrier particles with a solution of drug in water in a quantity of 1-3% by weight of solution to total mix.

2. A process according to claim 1 wherein the carrier is a soluble or an insoluble directly compressible pharmaceutically acceptable excipient.

3. A process according to claim 2 wherein the carrier is a soluble excipient selected from anhydrous lactose, lactose monohydrate and mannitol.

4. A process according to claim 3 wherein the mixture further comprises an acidic or alkaline excipient to improve chemical stability of the drug in the formulation.

5. A process according to any preceding claim which further comprises blending the mixture with further carrier and/or one or more additives selected from a disintegrant; an acidic or alkaline excipient to improve chemical stability of the drug in the formulation, a lubricant and a glidant.

6. A process according to claim 5 wherein the mix is blended with further carrier in a weight ratio of 1/4 to 1/40.

7. A process according to any preceding claim wherein the mixture is formulated into a unit dose presentation.

8. A process according to any preceding claim for formulating [R-(Z)]-α-(methoxyimino)-α-(1-azabicyclo [2.2.2]oct-3-yl)acetonitrile monohydrochloride.

9. A pharmaceutical composition comprising a drug, obtainable by the process of any preceding claim.

10. A pharmaceutical composition comprising a pharmaceutically acceptable carrier and [R-(Z)]-α-(methoxyimino)-α-(1-azabicyclo [2.2.2]oct-3-yl)acetonitrile monohydrochloride at a level of up to 0.1% by weight of drug to carrier, 0% volatile organic solvent and 0% binder.

11. A composition according to claim 10 wherein the carrier is a soluble or an insoluble directly compressible pharmaceutically acceptable excipient.

12. A composition according to claim 11 wherein the carrier is a soluble excipient selected from anhydrous lactose, lactose monohydrate and mannitol.

13. A composition according to any of claims 10 to 12 which also comprises one or more additives selected from a disintegrant, an acidic excipient to improve chemical stability of the drug in the formulation. a lubricant and a glidant.

14. A composition according to claim 13 which comprises lactose monohydrate, sodium dihydrogen citrate, colloidal silica and magnesium stearate.

15. A composition according to any of claims 10 to 14 formulated into a unit dose presentation.

16. A method of treatment and/or prophylaxis of dementia. which method comprises administering to the patient [(R-(Z)]-α-(methoxyimino)-α-(1-azabicyclo [2.2.2]oct-3- yl)acetonitrile monohydrochloride at a daily dose below 0.01 mg/kg.

17. The use of [R-(Z)]-α-(methoxyimino)-α-(1-azabicyclo [2.2.2]oct-3-yl)acetonitrile monohydrochloride in the manufacture of a medicament for the treatment and/or prophylaxis of dementia at a daily dose below 0.01 mg/kg.

18. A pharmaceutical composition for the treatment and/or prophylaxis of dementia which comprises [R-(Z)]-α-(methoxyimino)-α-(1-azabicyclo [2.2.2]oct-3-yl)acetonitrile monohydrochloride at a unit dose suitable for administration at a daily dose below 0.01 mg/kg, and a pharmaceutically acceptable carrier.

19. A method, use or composition according to claim 16, 17 or 18 wherein the daily dose is 0.003 mg/kg or less.

20. A method, use or composition according to claim 19 wherein the daily dose is 0.0001-0.003 mg/kg.

21. A method, use or composition according to claim 20 wherein the daily dose is 0.00035-0.003 mg/kg.

22. A method, use or composition according to claim 20 wherein the daily dose is 0.0007-0.003 mg/kg.

23. A method, use or composition according to claim 20 wherein the daily dose is 0.0001-0.0007 mg/kg.

24. A method, use or composition according to claim 20 wherein the daily dose is 0.00035-0.002 mg/kg.

25. A method, use or composition according to claim 16, 17 or 18 wherein the compound is presented in a unit dose of 5, 12.5, 25, 50 or 75 μg, administered twice daily or, in the case of 50 μg, once daily.

26. A pharmaceutical composition according to claim 9 as dependent on claim 8 or according to any of claims 10 to 15, in unit dose form selected from the range 5-125 μg per unit dose.

27. A pharmaceutical composition according to claim 26 comprising 5, 12.5, 25, 50 or 75 μg per unit dose.

28. A pharmaceutical composition according to claim 9, according to any of claims 10 to 15 or according to claim 26 or 27 for use as an active therapeutic substance.

29. A pharmaceutical composition according to claim 9 as dependent on claim 8, according to any of claims 10 to 15 or according to claim 26 or 27 for use in the treatment and/or prophylaxis of dementia.

30. A method of treatment and/or prophylaxis of dementia, which method comprises administering to the patient an effective amount of a composition according to claim 29.

31. Use of a pharmaceutical composition according to claim 29 in the manufacture of a medicament for the treatment and/or prophylaxis of dementia.
